(19) United States
(12) Patent Application Publication
(10) Pub. No.: US 2020/0321869 A1
(43) Pub. Date: Oct. 8, 2020

(54) DC/DC CONVERTER AND ULTRASONIC DIAGNOSTIC APPARATUS

(71) Applicant: CANON MEDICAL SYSTEMS CORPORATION, Otawara-shi (JP)

(72) Inventors: Yuji KUWANA, Otawara (JP); Shuta Fujiwara, Nasushiobara (JP)

(73) Assignee: CANON MEDICAL SYSTEMS CORPORATION, Otawara-shi (JP)

(21) Appl. No.: 16/802,634

(22) Filed: Feb. 27, 2020

(30) Foreign Application Priority Data
Feb. 28, 2019 (JP) 2019-035452

(51) Int. Cl. H02M 3/158; A61B 8/00; H02M 1/38; H03K 17/16

(52) U.S. Cl. CPC H02M 3/1582; A61B 8/56; H02M 3/1588; H03K 17/161; H02M 1/38

(57) ABSTRACT

In one embodiment, a DC/DC converter includes: a first switching element, a diode connected to the first switching element, an inductor connected to at least one of the first switching element and the diode, and a circuit connected in parallel to the first switching element, the circuit including a second switching element. A current flowing through the circuit when the second switching element is on is smaller than the current flowing through the first switching element when the first switching element is on, and the second switching element is turned on a lead time before the timing at which the first switching element is turned on.

### CROSS-REFERENCE TO RELATED APPLICATION

This application is based upon and claims the benefit of priority from Japanese Patent Application No. 2019-035452, filed on Feb. 28, 2019, the entire contents of which are incorporated herein by reference.

### FIELD

Embodiments described herein relate generally to a DC/DC converter and an ultrasonic diagnostic apparatus.

### BACKGROUND

In medical image diagnostic apparatuses such as an ultrasonic diagnostic apparatus, a CT apparatus, and an MRI apparatus, it is required to generate a medical image with high resolution and high SNR. Therefore, noise generated inside the apparatus is needed to be suppressed. In addition, even in an apparatus other than the medical image diagnostic apparatus, for example, an information processing apparatus such as a communication apparatus or a computer, it is necessary to minimize noise generated inside the apparatus.

One of the noise sources inside the apparatus is a direct current/direct current (DC/DC) converter. A DC/DC converter is a device that changes a direct current (DC) voltage to another DC voltage. In many apparatuses that use a 50 Hz/60 Hz commercial alternating current (AC) power supply as a main power supply, the commercial AC power is once converted to DC of a predetermined voltage by a rectifier circuit. Then, by using the DC/DC converter, the DC voltage is converted to a specified voltage value for respective operating power supplies of various devices or various integrated circuits in the apparatus.

Among the DC/DC converters, while there is a linear DC/DC converter such as a three-terminal regulator, a switching DC/DC converter is widely used from the viewpoint of conversion efficiency.

In the switching DC/DC converter, a spike-like current generated by a switching operation is a main noise source. Therefore, conventionally, a circuit for suppressing the spike-like noise or a device for reducing the generation of the noise itself has been developed.

Meanwhile, in an image diagnostic apparatus such as an ultrasonic diagnostic apparatus, a technique for improving the image quality has been developed, and accordingly, a demand for noise reduction of the DC/DC converter has been increased.

### BRIEF DESCRIPTION OF THE DRAWINGS

In the accompanying drawings:

FIG. 1 is a diagram showing a circuit configuration example of a conventional DC/DC converter;

FIG. 2 is an operation timing chart of a conventional DC/DC converter in a steady state;

FIG. 3 is a diagram showing a circuit configuration example of the DC/DC converter according to the first embodiment;

FIG. 4 is an operation timing chart in a steady state of the DC/DC converter of the first embodiment;

FIG. 5 is a diagram illustrating a circuit configuration example of the DC/DC converter according to a first modification of the first embodiment;

FIG. 6 is a diagram illustrating a circuit configuration example of the DC/DC converter according to a second modification of the first embodiment;

FIG. 7 is a diagram illustrating an example of a circuit configuration of the DC/DC converter according to a third modification of the first embodiment;

FIG. 8 is an operation timing chart in a steady state of the DC/DC converter according to the third modification of the first embodiment;

FIG. 9 is a diagram illustrating a circuit configuration example of the DC/DC converter according to a second embodiment;

FIG. 10 is a diagram illustrating a circuit configuration example of the DC/DC converter according to a third embodiment; and

FIG. 11 is a block diagram showing a configuration example of an ultrasonic diagnostic apparatus.

### DETAILED DESCRIPTION

(DC/DC Converter)

In one embodiment, a DC/DC converter includes: a first switching element, a diode connected to the first switching element, an inductor connected to at least one of the first switching element and the diode, and a circuit connected in parallel to the first switching element, the circuit including a second switching element. A current flowing through the circuit when the second switching element is on is smaller than the current flowing through the first switching element when the first switching element is on, and the second switching element is turned on a lead time before the timing at which the first switching element is turned on.

Hereinafter, the present embodiment will be described with reference to the accompanying drawings. Before that, the problems of the conventional DC/DC converter will be briefly described.

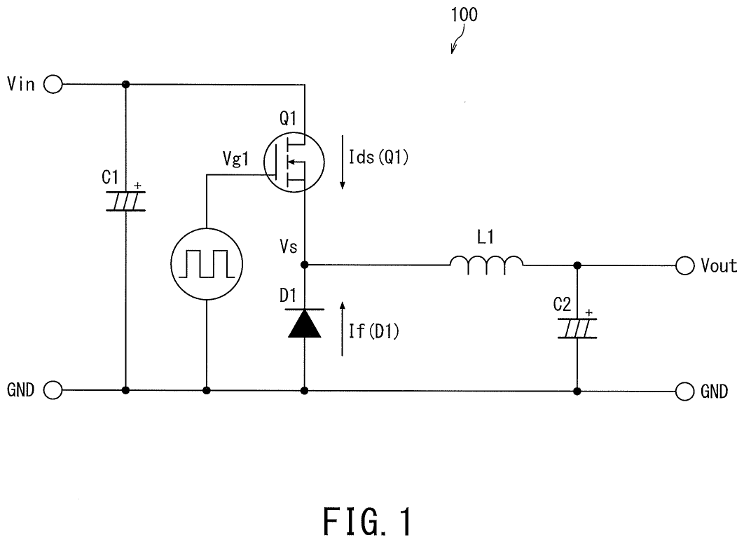

FIG. 1 is a diagram showing a typical circuit example of a conventional DC/DC converter 100. There are three types of non-insulated DC/DC converters. The first type is a buck converter in which the output is converted to a voltage lower than the input. The second type is a boost converter in which the output is converted to a higher voltage than an input. The third type is a buck-boost converter whose output can be converted to both lower and higher voltages than the input.

FIG. 1 shows a circuit example of a conventional buck converter 100 among these three types. Problems related to the spike noise described later are common to the above three types. Therefore, by referring to the conventional DC/DC converter (buck converter) 100 shown in FIG. 1, a schematic operation of this circuit and problems related to spike noise will be described.

In the DC/DC converter 100, the input voltage Vin is applied between the input terminal “Vin” and “GND (ground)”, and the voltage of Vout is output between the output terminal “Vout” and “GND”.

The main elements included in the conventional DC/DC converter 100 are three elements: a switching element Q1, a diode D1, and an inductor L1. In addition, smoothing capacitors C1 and C2 are provided in parallel with the input terminal and the output terminal, respectively.

The switching element Q1 is, for example, an FET (Field Effect Transistor), and switches the current Ids (Q1) flowing from the drain to the source according to the on/off of a pulse signal applied to the gate. That is, when the pulse signal is on, the switching element Q1 is turned on (closed), and the current Ids (Q1) flows between the drain and the source. On the other hand, when the pulse signal is off, the switching element Q1 is turned off (open), and no current flows between the drain and source.

The diode D1 is also called a freewheel diode. When the pulse signal is on, the diode D1 is reverse-biased, and the current flowing through the switching element Q1 does not flow to the ground but flows into the inductor L1. On the other hand, when the pulse signal is off, the bias becomes forward bias, and the forward current If (D1) flowing from the anode side (ground side) of the diode D1 to the cathode side does not flow to the switching element Q1 side but flows into the inductor L1.

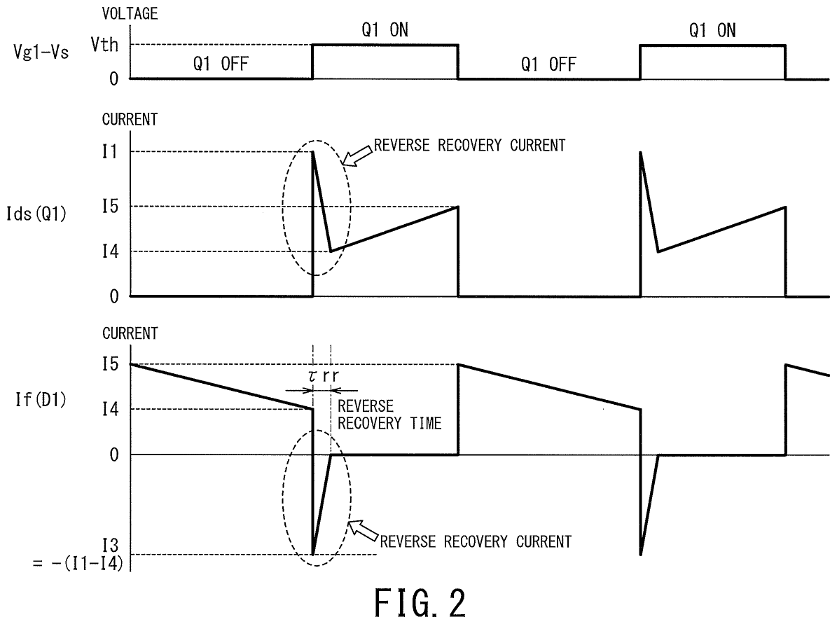

FIG. 2 is a timing chart schematically showing voltage and current waveforms for explaining the operation of the DC/DC converter 100 in a steady state.

The upper part of FIG. 2 shows a timing chart of the voltage (Vg1-Vs) of the pulse signal applied between the drain and source of the switching element Q1. When the voltage (Vg1-Vs) of the pulse signal exceeds a predetermined threshold voltage Vth (that is, when the pulse signal turns on), the switching element Q1 also turns on (that is, closes). On the other hand, when the voltage (Vg1-Vs) of the pulse signal becomes lower than the predetermined threshold voltage Vth (that is, when the pulse signal turns off), the switching element Q1 also turns off (that is, opens).

The middle part of FIG. 2 shows a timing chart of the current Ids (Q1) flowing through the switching element Q1, which changes in conjunction with the ON/OFF of the pulse signal. Similarly, the lower part of FIG. 2 shows a timing chart of the current If (D1) flowing through the diode D1, which changes in conjunction with the ON/OFF of the pulse signal.

The spike-shaped current common to the current Ids (Q1) and the current If (D1) is called a reverse recovery current. The reverse recovery current is a current that flows in a direction opposite to the forward current for a short period of time, without becoming immediately zero, when the diode D1 switches from a forward bias state to a reverse bias state. The period during which the reverse recovery current flows is called the reverse recovery time τrr.

In the DC/DC converter 100 shown in FIG. 1, while the pulse signal is off, the diode D1 is in a forward bias state, and the forward current If (D1) is flowing. When the pulse signal is turned on from this state, the diode D1 changes from a forward bias to a reverse bias, resulting in that a current in a negative direction (i.e., a direction from the cathode to the anode) flows in the diode D1, that is, the above-mentioned reverse recovery current flows in the diode D1, as shown in the lower part of FIG. 2. After the pulse signal is turned on, the reverse recovery current flows in a direction from the input voltage terminal Vin to the ground side through the switching element Q1 and the diode D1 for a reverse recovery time τrr, and thereafter becomes zero.

Note that, at the timing when the pulse signal switches from ON to OFF (that is, when the diode D1 switches from reverse bias to forward bias), no reverse recovery current occurs. Further, during the period in which the pulse signal is OFF, a forward current If (D1) that decreases almost linearly from the current value I5 to I4 flows through the diode D1, as shown in the lower part of FIG. 2. This forward current If (D1) flows into the inductor L1 from the cathode of the diode D1.

In the meantime, in conjunction with the reverse recovery current flowing through the diode D1, the reverse recovery current having the same current value flows from the drain to the source of the switching element Q1.

During the period when the switching element Q1 is on after the reverse recovery time τrr has elapsed, the current Ids (Q1) flowing through the switching element Q1 increases almost linearly from, for example, a predetermined current I4 to I5. This current Ids (Q1) flows into the inductor L1 from the source of the switching element Q1. When the switching element Q1 is turned off, the current Ids (Q1) becomes zero.

As described above, the current Ids (Q1) that increases from the current value I4 to I5 flows into the inductor L1 while the pulse signal is on, and the current If (D1) that decreases from the current value I5 to I4 also flows into the inductor L1 while the pulse signal is off.

Note that the above-described reverse recovery current flows through the switching element Q1 and the diode D1 in addition to the trapezoidal current whose current value increases from I4 to I5 or the current value decreases from I4 to I5. Since this reverse recovery current has a large value that cannot be ignored, it becomes a spike noise source. Further, ringing may occur immediately after the reverse recovery current, and this ringing also becomes a noise source.

In addition, for a DC/DC converter that handles a large amount of power, a large reverse recovery current also causes power loss that cannot be ignored.

The above-described reverse recovery current is caused by the potential property of the diode D1, and its effect can be reduced by shortening the reverse recovery time τrr. As a diode having a short reverse recovery time τrr, a diode called a Fast Recovery Diode or a Schottky barrier diode are well known. Therefore, in DC/DC converters used in medical image processing apparatuses, these low-noise diodes have conventionally been used.

However, even in a Schottky barrier diode whose reverse recovery current is said to be almost zero, it has been said that a considerable amount of reverse recovery current actually flows. For example, it is known that although the reverse recovery time τrr is as short as about several nanoseconds, the reverse recovery current can be as large as about 100 to 200 amperes, or in some cases, about several hundred amperes.

Thus, the DC/DC converter 1 of each embodiment described below has a circuit configuration that can reduce the above-described reverse recovery current without increasing the circuit scale so much.

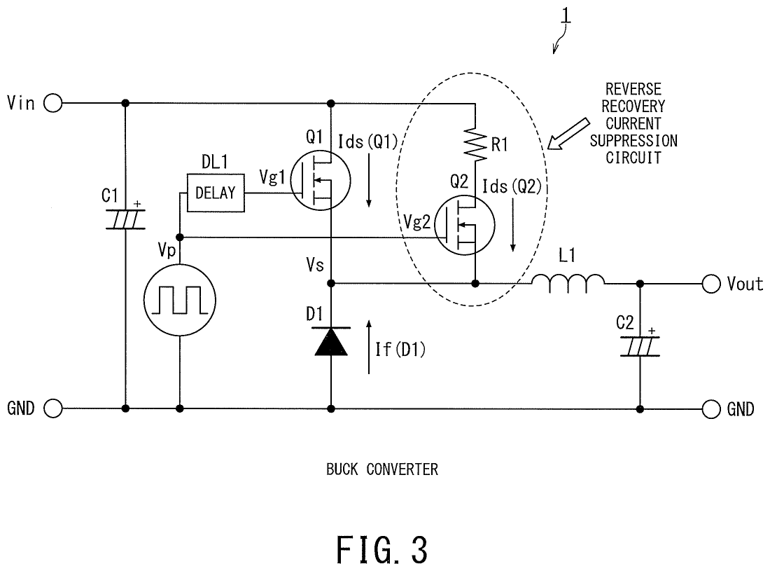

FIG. 3 is a diagram illustrating a circuit example of the DC/DC converter 1 according to the first embodiment. The DC/DC converter 1 of the first embodiment is configured as a buck converter. The difference between the DC/DC converter 1 according to the first embodiment and the conventional DC/DC converter 100 is that, in parallel with the switching element Q1 (hereinafter, referred to as a first switching element Q1), a reverse recovery current suppression circuit (that is, a noise suppression circuit) including a series circuit of a second switching element Q2 and the resistor R1 is provided.

A first pulse signal is applied to the gate of the first switching element Q1 as a gate voltage Vg1 from a pulse signal source Vp via a delay element DL1. On the other hand, the second pulse signal is applied to the gate of the second switching element Q2 from the pulse signal source Vp as the gate voltage Vg2 without delay.

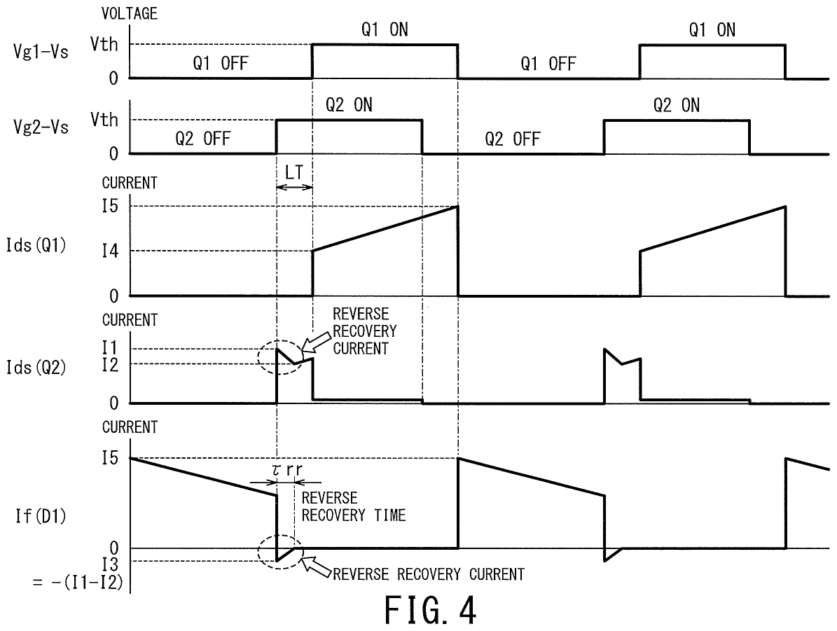

FIG. 4 is a timing chart schematically illustrating a voltage/current waveform for describing an operation in a steady state of the DC/DC converter 1 according to the first embodiment.

The first stage in FIG. 4 shows a timing chart of the voltage (Vg1-Vs) of the first pulse signal applied between the drain and the source of the first switching element Q1. When the voltage (Vg1-Vs) of the first pulse signal exceeds a predetermined threshold voltage Vth (that is, when the first pulse signal turns on), the switching element Q1 also turns on (that is, closes). Conversely, when the voltage (Vg1-Vs) of the first pulse signal becomes lower than the predetermined threshold voltage Vth (that is, when the first pulse signal turns off), the switching element Q1 also turns off (that is, opens).

The second stage in FIG. 4 shows a timing chart of the voltage (Vg2-Vs) of the second pulse signal applied between the drain and the source of the second switching element Q2. The waveform of the second pulse signal is similar to the waveform of the first pulse signal, and has a waveform preceding the first pulse signal by the lead time LT. When the voltage (Vg2-Vs) of the second pulse signal exceeds a predetermined threshold voltage Vth (that is, when the second pulse signal turns on), the switching element Q2 also turns on (that is, closes). Conversely, when the voltage (Vg2-Vs) of the second pulse signal becomes lower than the predetermined threshold voltage Vth (that is, when the second pulse signal turns off), the switching element Q2 also turns off (that is, opens).

The third stage in FIG. 4 is a timing chart of the current Ids (Q1) flowing through the switching element Q1, which changes in conjunction with the ON/OFF of the first pulse signal. The fourth stage in FIG. 4 is a timing chart of the current Ids (Q2) flowing through the switching element Q2, which changes in conjunction with the ON/OFF of the second pulse signal. And, the lowest stage in FIG. 4 is a timing chart of the current If (D1) flowing through the diode D1, which changes in conjunction with the respective ON/OFF of the first pulse signal and the ON/OFF of the second pulse signal.

As described above, the reverse recovery current occurs immediately after the diode D1 switches from the forward bias state to the reverse bias state. As shown in the circuit of FIG. 3, in the DC/DC converter 1 of the first embodiment, the reverse recovery current suppressing circuit including a series circuit of the second switching element Q2 and the resistor R1 is provided in parallel with the first switching element Q1. Therefore, while the first switching element Q1 and the second switching element Q2 are simultaneously turned off, the diode D1 is in a forward bias state, and the forward current If (D1) flows from the ground through the diode D1 to the inductor L1. As shown on the left side of the lowest stage in FIG. 4, the forward current If (D1) decreases almost linearly, for example, from the current value I5 during a period in which the first and second switching elements Q1 and Q2 are simultaneously turned off.

Here, the second switching element Q2 is turned on earlier than the first switching element Q1 by the lead time LT. Note that, when the second switching element Q2 is turned on, the diode D1 switches from the forward bias state to the reverse bias state. Immediately after this switching, a reverse recovery current flows during a reverse recovery period τrr.

In the DC/DC converter 1 according to the first embodiment, the second switching element Q2 is turned on first while the first switching element Q1 remains off. Therefore, the reverse recovery current from the input voltage terminal Vin to the ground side flows through the diode D1 through the series connection circuit (that is, the noise suppression circuit) including the resistor R1 and the second switching element Q2.

Accordingly, in the DC/DC converter 1 of the first embodiment, the reverse recovery current is limited by the resistor R1. As a result, the peak value of the reverse recovery current is greatly suppressed.

In the conventional DC/DC converter 100, there is only the switching element Q1 between the input voltage terminal Vin and the cathode of the diode D1. Further, the internal resistance when the switching element Q1 is on is very small. For this reason, the peak value of the reverse recovery current can be a very large value, for example, about 100 to 200 amperes, or several hundred amperes.

By contrast, in the DC/DC converter 1 according to the first embodiment, between the input voltage terminal Vin and the cathode of the diode D1, the first switching element Q1 and a series circuit including the resistor R1 and the second switching element Q2 are connected in parallel. For this reason, during the lead time LT after the second switching element Q2 is turned on and then until the first switching element Q1 is turned on, the combined resistance between the input voltage terminal Vin and the cathode of the diode D1 is substantially equal to the resistance R1, and the reverse recovery current is limited by the resistance R1.

As a result, as shown in the lowest stage of the bottom of FIG. 4, the reverse recovery current becomes I3 (=−(I1−I2)), which is much smaller than the reverse recovery current of the conventional DC/DC converter 100. The resistance value of the resistor R1 is set to, for example, about several ohms, or about 5 to 10 ohms. Therefore, if the voltage of the input voltage Vin is, for example, 10 volts and the resistance of the resistor R1 is, for example, 10 ohms, the peak value of the reverse recovery current becomes one ampere.

While the peak value of the reverse recovery current in the conventional DC/DC converter 100 becomes very large, for example, becomes about 100 to 200 amperes, the peak value of the reverse recovery current in the DC/DC converter 1 of the first embodiment is greatly reduced to about several amperes, for example, to about one ampere.

After the reverse recovery time τrr, the forward current If (D1) of the diode D1 becomes zero. Thereafter, when the first switching element Q1 switches from on to off, the diode D1 returns from the reverse bias state to the forward bias state. Then, a forward current If (D1) that gradually decreases almost linearly from the current value I5 flows through the diode D1.

In the meantime, during the period when the second switching element Q2 is ON and the first switching element Q1 is OFF, that is, during the lead time LT, in the second switching element Q2, a combined current obtained by adding an rising trapezoid shaped current having a time width LT and the reverse recovery current. After the combined current, a very small current, for example, a few milliamperes, flows through the second switching element Q2. This period is a period during which the first switching element Q1 and the second switching element Q2 are simultaneously turned on. Since the internal resistance, when the first switching element Q1 is ON, is sufficiently smaller than the resistance R1, while most of the current (Ids (Q1)) flows through the first switching element Q1, the current Ids (Q2) flowing through the second switching element Q2 is very small, such as a few milliamperes.

The time difference between the first pulse signal and the second pulse signal, that is, the lead time LT, is set so that a reverse recovery current flows to the series circuit of the second switching element Q2 and the resistor R1 during this period LT. Therefore, the lead time LT is preferably the same time as the reverse recovery time τrr of the diode D1, or a time obtained by adding a predetermined margin to the reverse recovery time τrr, for example, a margin of 10% to 50% of the reverse recovery time τrr.

According to the DC/DC converter 1 of the first embodiment described above, a series circuit of the second switching element Q2 and the resistor R1 is provided in parallel with the first switching element Q1, and a delay element DL1 connected to the gate of the first switching element Q1 is also provided. By adding such a relatively small circuit to the conventional DC/DC converter 100, the peak value of the reverse recovery current can be significantly reduced. As a result, spike-like noise caused by the reverse recovery current can be significantly suppressed.

Further, the period during which the current flows in the series circuit of the second switching element Q2 and the resistor R1 is only a small part of the entire time, and the current value is also small. Therefore, the increase in power consumption of the DC/DC converter 1 is small, and the conversion efficiency is not significantly impaired.

In addition, as described above, in the case of a DC/DC converter that handles a large amount of power, a large reverse recovery current causes power loss to be insignificant. By contrast, according to the DC/DC converter 1 of the first embodiment, since the reverse recovery current is reduced, the power conversion efficiency is also improved.

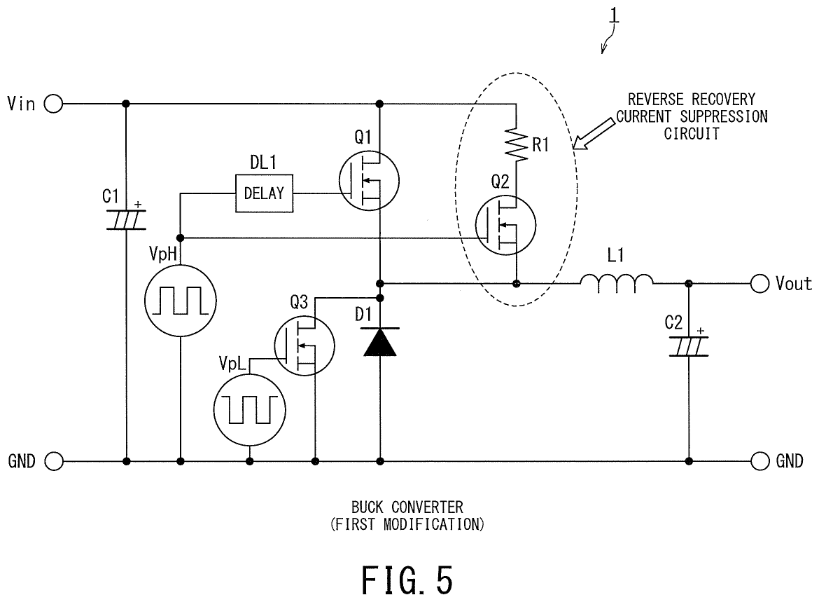

FIG. 5 is a diagram illustrating a circuit configuration example of the DC/DC converter 1 according to the first modification of the first embodiment. The first modification of the first embodiment is different from the first embodiment in that the first modification is configured as a so-called synchronous DC/DC converter 1. They are common in that they are buck converters.

The synchronous DC/DC converter 1 has a third switching element Q3, and applies a third pulse signal, which has characteristics reverse to those of the first pulse signal applied to the gate of the first switching element Q1, to the gate of the third switching element Q3. In this configuration of the synchronous DC/DC converter 1, a circuit for generating the third pulse signal and the additional switching element Q3 are required, and thus, the circuit scale is larger than that of the first embodiment. However, since the forward voltage of the third switching element Q3 is smaller than the forward voltage of the diode D1 shown in FIG. 3, there is an advantage that the conversion efficiency is high.

Also in the circuit of the first modification, a series circuit of the second switching element Q2 and the resistor R1 is provided in parallel with the first switching element Q1, and a delay element DL1 is provided at the gate of the first switching element Q1, which is the same as that of the first embodiment (FIG. 3). Therefore, the same effects as those of the first embodiment can be obtained in the first modification of the first embodiment.

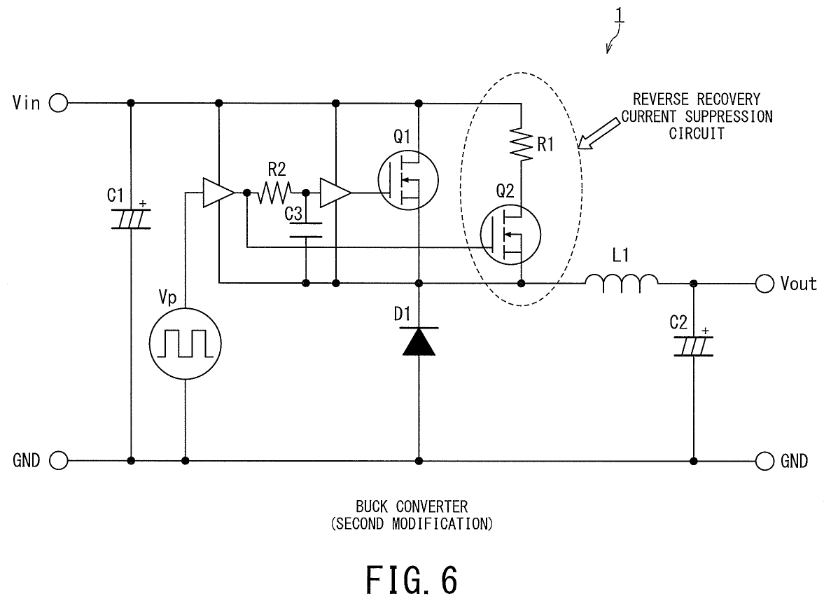

FIG. 6 is a diagram illustrating a circuit configuration example of the DC/DC converter 1 according to the second modification of the first embodiment. In the second modification of the first embodiment, the delay element DL1 in the first embodiment (FIG. 3) is replaced with a CR delay circuit including a resistor R2 and a capacitor C3 and inverters sandwiching the CR delay circuit. The value of the lead time LT can be adjusted by the time constant of the resistor R2 and the capacitor C3. Other configurations in the second modification of the first embodiment are the same as those of the first embodiment. Therefore, similarly to the first embodiment, also in the second modification of the first embodiment, the effects of reducing the reverse recovery current and reducing the spike-like noise obtained therewith are obtained.

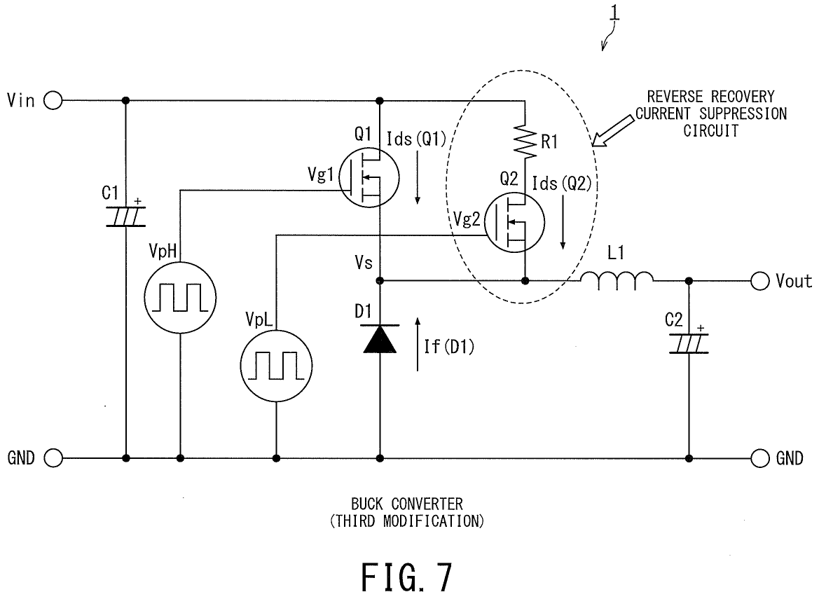

FIG. 7 is a diagram illustrating an example of a circuit configuration of the DC/DC converter 1 according to the third modification of the first embodiment. In the third modification of the first embodiment, a first pulse signal source VpH for generating a first pulse signal and a second pulse signal source VpL for generating a second pulse signal are separately provided. This is different from the first embodiment shown in FIG. 3. The first pulse signal generated by the first pulse signal source VpH is applied to the gate of the first switching element Q1, while the second pulse signal generated by the second pulse signal source VpL is applied to the gate of the second switching element Q2. With such a configuration, in the third modification, the timing for turning on and off the first switching element Q1 and the timing for turning on and off the second switching element Q2 can be independently controlled.

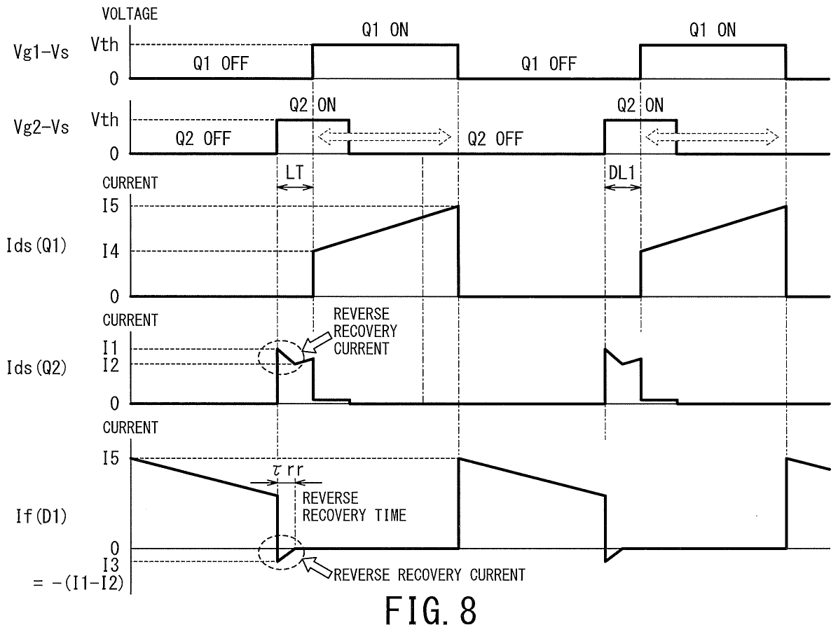

FIG. 8 is a timing chart schematically showing voltage/current waveforms for explaining an operation in a steady state of the DC/DC converter 1 according to the third modification of the first embodiment. The timing chart is the same as the timing chart of the first embodiment (FIG. 4) except for the ON/OFF timing of the second switching element Q2 shown in the second stage of FIG. 8.

In the third modification of the first embodiment, as in the first embodiment, the ON timing of the second switching element Q2 is applied earlier than the ON timing of the first switching element Q1 by the lead time LT. On the other hand, the timing at which the second switching element Q2 is turned off can be set to an arbitrary time during the period when the first switching element Q1 is on. In the third modification of the first embodiment, the same effect as that of the first embodiment can be obtained.

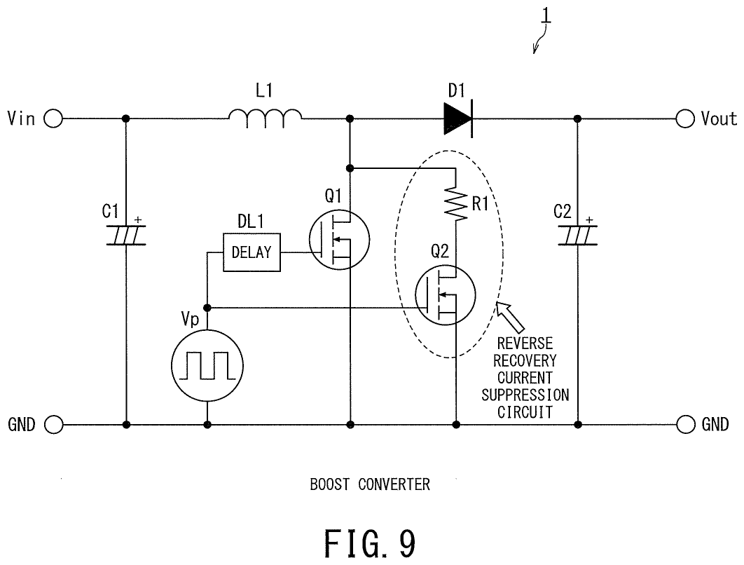

FIG. 9 is a diagram illustrating a circuit configuration example of the DC/DC converter 1 according to the second embodiment. The DC/DC converter 1 according to the second embodiment is configured as a boost converter.

As can be seen by comparing FIGS. 9 and 3, between the boost converter 1 (FIG. 9) of the second embodiment and the buck converter 1 (FIG. 3) of the first embodiment, the arrangement relationship of the inductor L1, the first switching element Q1, and the diode D1 is different. With the arrangement of the inductor L1, the first switching element Q1, and the diode D1 shown in FIG. 9, the DC/DC converter 1 (the boost convertor) that converts the output voltage to a voltage higher than the input voltage can be obtained.

Meanwhile, with respect to the point that a series circuit of the second switching element Q2 and the resistor R1 is provided in parallel with the first switching element Q1, and the delay element DL1 is provided at the gate of the first switching element Q1, there is no difference between the first embodiment and the respective modifications, and the second embodiment.

Also in the second embodiment, a reverse recovery current occurs immediately after the diode D1 changes from the forward bias state to the reverse bias state. However, since the reverse recovery current flows to the series circuit of the second switching element Q2 and the resistor R1 during the lead time LT, the current is limited by the resistor R1. As a result, the reverse recovery current can be suppressed. As described above, the same effects as in the first embodiment can be obtained in the second embodiment.

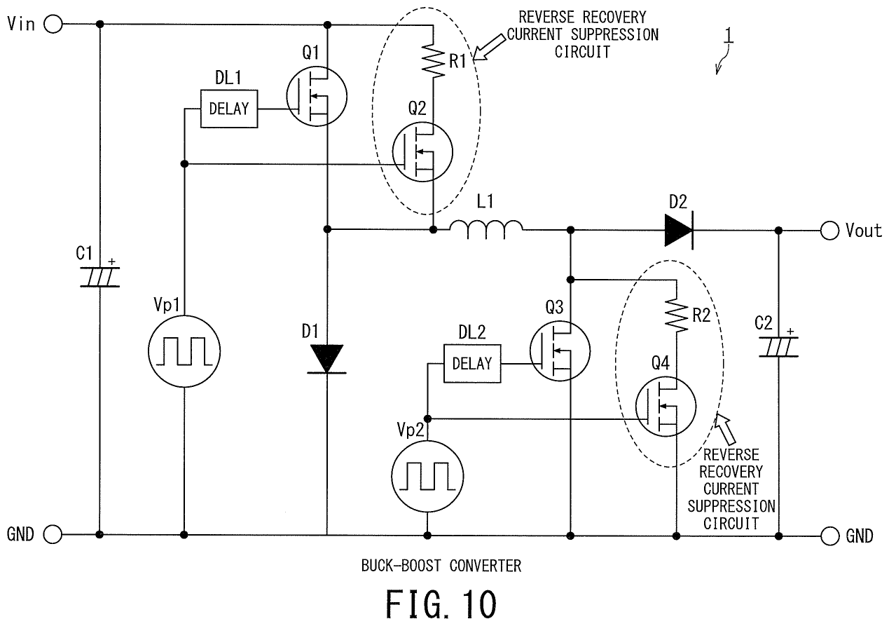

FIG. 10 is a diagram illustrating a circuit configuration example of the DC/DC converter 1 according to the third embodiment. The DC/DC converter 1 of the third embodiment is configured as a buck-boost converter. The buck-boost converter 1 includes a circuit configuration of the buck converter 1 of the first embodiment (FIG. 3) and a circuit configuration of the boost converter 1 of the second embodiment (FIG. 9).

As can be seen from FIG. 10, the circuit configuration of the buck-boost converter 1 is such that the first switching element Q1 and the first diode D1 are arranged to form a buck converter on the input side of the inductor L1. In addition, a series circuit of a second switching element Q2 and a first resistor R1 is provided in parallel with the first switching element Q1, and a first delay element DL1 is provided at the gate of the first switching element Q1. With such a circuit configuration, as in the first embodiment, the reverse recovery current generated in the first diode D1 can be suppressed.

On the other hand, on the output side of the inductor L1 of the buck-boost converter 1, the third switching element Q3 and the second diode D2 form an arrangement of a boost converter. Further, a series circuit of a fourth switching element Q4 and a second resistor R2 is provided in parallel with the third switching element Q3, and a second delay element DL2 is provided at the gate of the third switching element Q3. With such a circuit configuration, the reverse recovery current generated in the second diode D2 can be suppressed as in the second embodiment. As described above, also in the third embodiment, the same effect as in the first embodiment can be obtained.

(Ultrasonic Diagnostic Equipment)

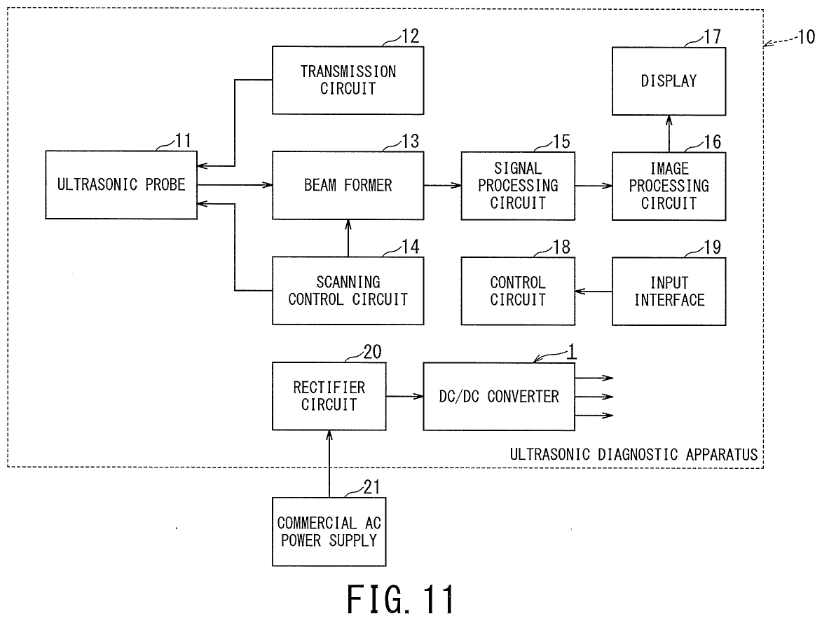

FIG. 11 is a block diagram showing a configuration example of an ultrasonic diagnostic apparatus 10 including the DC/DC converter 1 of each of the above-described embodiments and modifications.

The ultrasonic diagnostic apparatus 10 includes, for example, an ultrasonic probe 11, a transmission circuit 12, a beam former 13, a scanning control circuit 14, a signal processing circuit 15, an image processing circuit 16, a display 17, a control circuit 18, and an input interface 19.

The ultrasonic probe 11 transmits the ultrasonic pulse generated by the transmission circuit 12 to the object, and receives a reflected wave from the object. The beam former 13 forms a receiving beam by performing weighted addition processing on the reflected wave received by the ultrasonic probe 11. Further, the beam former 13 scans the receiving beam according to a control signal from the scanning control circuit 14. The signal processing circuit 15 performs signal processing such as logarithmic detection processing, correlation processing, and Doppler processing on the received signal subjected to the beam forming processing. The image processing circuit 16 generates an image such as a B-mode image, a Doppler mode image, a color Doppler mode image, etc., based on the signal after the signal processing and information such as the scanning angle. The display 17 displays the generated image. The input interface 19 is an operation device such as a mouse, a trackball, and a touch panel. The control circuit 18 generates a control signal for controlling each circuit and/or device of the ultrasonic diagnostic apparatus 10 based on operation information and/or data input through the input interface 19.

The rectifier circuit 20 rectifies the commercial AC power supply 21 input from the outside, and converts it into a DC voltage. Then, the DC/DC converter 1 converts this DC voltage into a predetermined DC voltage required for each circuit and/or device of the ultrasonic diagnostic apparatus 10.

By using the circuit of each of the above-described embodiments and modifications as the DC/DC converter 1, it is possible to suppress the reverse recovery current generated by switching, and to reduce spike noise and ringing noise. As a result, the ultrasonic diagnostic apparatus 10 can generate a high-quality medical image with less noise.

Note that the term “reverse recovery current suppressing circuit”, “noise suppressing circuit”, or “series circuit” described in this specification is an example of the “circuit” described in the claims.

According to at least one embodiment described above, the spike-like noise and/or ringing noise of the DC/DC converter used in various apparatus such as an ultrasonic diagnostic apparatus can be reduced, without significantly impairing power conversion efficiency.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A DC/DC converter comprising:
a first switching element;
a diode connected to the first switching element;
an inductor connected to at least one of the first switching element and the diode; and
a circuit connected in parallel to the first switching element, the circuit including a second switching element;
wherein a current flowing through the circuit when the second switching element is on is smaller than the current flowing through the first switching element when the first switching element is on, and
the second switching element is turned on a lead time before the timing at which the first switching element is turned on.

2. The DC/DC converter according to claim 1,
wherein the circuit comprises a resistor connected in series with the second switching element.

3. The DC/DC converter according to claim 2,
wherein an input voltage is applied to one end of the first switching element and one end of the resistor,
another end of the first switching element is connected to a cathode of the diode,
another end of the resistor is connected to one end of the second switching element,
another end of the second switching element is connected to the cathode of the diode,
an anode of the diode is connected to ground, and
another end of the second switching element and the cathode of the diode are connected to the inductor.

4. The DC/DC converter according to claim 2,
wherein one end of the first switching element and one end of the second switching element are connected to ground,
another end of the first switching element is connected to an anode of the diode,
another end of the second switching element is connected to one end of the resistor, and
another end of the resistor is connected to the anode of the diode and the inductor.

5. The DC/DC converter according to claim 2,
wherein the lead time is a same time as a reverse recovery time of the diode, or a time obtained by adding a margin time to the reverse recovery time.

6. The DC/DC converter according to claim 2,
wherein the first switching element is turned on and off by a first pulse signal, and the second switching element is turned on and off by a second pulse signal.

7. The DC/DC converter according to claim 6, further comprising a delay element,
wherein the first pulse signal is a signal obtained by delaying the second pulse signal by the lead time with the delay element.

8. The DC/DC converter according to claim 6, further comprising an RC delay circuit configured by a resistor and a capacitor,
wherein the first pulse signal is a signal obtained by delaying the second pulse signal by the lead time with the RC delay circuit.

9. The DC/DC converter according to claim 6,
wherein a timing at which the second switching element is turned on by the second pulse signal is set earlier by the lead time than a timing at which the first switching element is turned on by the first pulse signal, and
a timing at which the second switching element is turned off by the second pulse signal is set at any time during a period after the first switching element is turned on by the first pulse signal before the first switching element is turned off by the first pulse signal.

10. The DC/DC converter according to claim 2,
wherein the DC/DC converter is configured as a non-insulated type converter.

11. The DC/DC converter according to claim 10,
wherein the DC/DC converter is configured as a boost converter.

12. The DC/DC converter according to claim 10,
wherein the DC/DC converter is configured as a buck converter.

13. The DC/DC converter according to claim 10,
wherein the DC/DC converter is configured as a buck-boost converter.

14. An ultrasonic diagnostic apparatus comprising the DC/DC converter according to claim 2.
